SEQUENCE LISTING (2 sequences)

SEQ ID NO: 1 - DNA, 50 bp, Homo sapiens
  misc_feature (42)..(42): n is a, c, g, or t
tcgtcggcagcgtcagatgtgtataagagacagcctacgggnggcwgcag

SEQ ID NO: 2 - DNA, 55 bp, Homo sapiens
gtctcgtgggctcggagatgtgtataagagacaggactachvgggtatctaatcc

(19) United States
(12) Patent Application Publication
(10) Pub. No.: US 2019/0000759 A1
(43) Pub. Date: Jan. 3, 2019

(54) ANTIMICROBIAL GEL CONTAINING SILVER NANOPARTICLES

(71) Applicant: OTAGO INNOVATION LIMITED, Dunedin (NZ)

(72) Inventors: Carla Joy MELEDANDRI, Dunedin (NZ); Donald Royden SCHWASS, Dunedin (NZ); Gemma Claire COTTON, Norfolk (GB); Warwick John DUNCAN, Dunedin (NZ)

(73) Assignee: OTAGO INNOVATION LIMITED, Dunedin (NZ)

(21) Appl. No.: 15/766,045

(22) PCT Filed: Oct. 4, 2016
(86) PCT No.: PCT/NZ2016/050162

§ 371 Date: Apr. 5, 2018

Related U.S. Application Data
(60) Provisional application No. 62/237,291, filed on Oct. 5, 2015.

(51) Int. Cl. A61K 9/06; A61K 33/38; A61P 31/04; A61P 31/10; A61K 9/51; A61K 47/12; A61K 47/36; A61P 1/02

(52) U.S. Cl. CPC A61K 9/06; A61K 33/38; A61P 31/04; A61P 1/02; A61K 9/51; A61K 47/12; A61K 47/36; A61P 31/10

(57) ABSTRACT

A gel comprising nano-sized particles of metallic silver (Ag), a polymer comprising carboxylate groups, carboxylate molecules comprising at least one group capable of binding to Ag, and metal ions, where the gel is useful as a topically applied antimicrobial agent.

### CROSS REFERENCE TO RELATED APPLICATIONS

This application is a 371 national phase of PCT/NZ2016/050162, filed Oct. 4, 2016, which claims the benefit of the filing date of U.S. Application No. 62/237,291, filed Oct. 5, 2015, the disclosures of which are incorporated, in their entirety, by this reference.

### TECHNICAL FIELD

The invention relates to an antimicrobial gel containing silver nanoparticles. In particular, the invention relates to a gel having a structure comprising silver nanoparticles bound to each other and to polymer chains through functionalised alkylcarboxylate molecules and metal ions. The invention further relates to the use of the gel as an antimicrobial agent for treating or preventing bacterial and fungal infections.

### BACKGROUND OF THE INVENTION

Many antimicrobial therapeutic agents are intended for oral or intravenous administration. However, administration by such methods is not effective in all situations. For example, an antimicrobial agent may need to be applied topically at the site of infection or site exposed to a high risk of infection. Periodontitis (gum disease) is one disease where topical application of an antibacterial agent is more effective. Denture stomatitis is an inflammatory condition affecting the oral mucosa beneath the fitting surface of dentures, and in over 90% of cases Candida is involved. Treatment requires the topical application of an antifungal agent. Other situations where the topical application of an antimicrobial agent is necessary or preferred include topical hemostatic agents (Achneck, H. E. et al., Annals of Surgery, 2010, 251, 217-228), cutaneous/burn wound healing (Abdelbary, G. A. et al., European Journal of Pharmaceutics and Biopharmaceutics, 2014, 86, 178-189; Kant, V. et al., Acta Histochem., 2014, 116, 5-13), antimicrobial treatment of skin lesions (Zhang, L. et al., ACS Nano, 2014, 8, 2900-2907; Jodar et al., Journal of Pharmaceutical Sciences, 2015, 104, 2241-2254), and the topical delivery of an antifungal agents to treat, for example, candidiasis, including, but not limited to oral, mammary, skin, vaginal, etc. (Jain, S. et al. Drug Deliv., 2010, 17, 443-451; Sobel, J. D. et al., Am. J. Obstet. Gynecol., 2003, 189, 1297-1300).

Periodontitis is an inflammatory disease caused by bacterial infection of the supporting tissue around the teeth. This causes loss of teeth, and has been linked to serious conditions including cardiovascular disease, stroke and pre-term birth. Missing teeth may be replaced using titanium dental implant screws and crowns. This highly effective treatment is common, but is expensive and is also vulnerable to gum disease. Peri-implant mucositis is an inflammatory lesion confined to the soft gum tissue around implants, while peri-implantitis also affects the supporting bone and causes painful disfiguring infections, resulting in the loosening of the implant and causing it to eventually fall out. Peri-implantitis is found in about 40% of individuals with implants, and peri-implant mucositis in about 50%. The causative bacteria are the same as those responsible for periodontitis and tooth loss. Current multimodal treatment strategies for both periodontitis and peri-implantitis involve the physical disruption of biofilms and chemotherapy with disinfectants and antibiotics, all with limited success and only capable of slowing the disease process at best.

Metallic silver (Ag) nanoparticles (NPs) exhibit strong antimicrobial activity against both Gram-positive and Gram-negative bacteria associated with these disease processes without the occurrence of resistance. The Ag NPs are typically applied directly to the infection site (unlike systemically delivered antibiotics which have been proven to have effectiveness for the treatment of peri-implantitis).

In contrast to disinfecting rinses such as iodine or chlorhexidine, it is anticipated that sustained antimicrobial activity may be possible where the antimicrobial agent is delivered via a gel structure. A gel is more likely to be retained within pocket infection spaces for a longer period of time.

Alginate, or alginic acid, is a naturally-occurring hydrophilic, linear copolymer comprising D-mannuronate (M) and L-guluronate (G) units. Alginate and alginate-based materials, commonly in a hydrogel form, have been developed for biomedical applications including tissue engineering, wound dressings and drug delivery. Hydrogels are a class of materials consisting of an interpenetrating, three-dimensional network of cross-linked hydrophilic polymer chains capable of accumulating large amounts of water or biological fluids, causing the materials to swell. Hydrogels of alginate can be formed through physical (e.g., ionic interactions) and/or covalent cross-linking of adjacent polymer chains.

The most common method for producing alginate hydrogels is through ionic cross-linking. Alginate selectively binds divalent metal ions (e.g. Ba²⁺, Sr²⁺ and Ca²⁺) with L-guluronate (G). Thus, a gel network can be formed when G-blocks of adjacent polymer chains are cross-linked by divalent cations through interactions with the carboxylate moieties.

The incorporation of silver ions and/or Ag NPs into alginate solutions and/or gels has been attempted using a variety of methods, with varying degrees of success. Ag NPs have been synthesised in alginate solutions (not gels) using gamma irradiation to give a colloidal Ag NP dispersion with the Ag NPs stabilised by the alginate polymer. Aqueous suspensions of alginate-stabilised Ag NPs have been produced by the reduction of AgNO₃ by NaBH₄ in the presence of a viscous solution of sodium alginate. After freeze-drying, exposure to CaCl₂ to induce cross-linking, and further processing, an insoluble alginate-Ag NP composite sponge formed and was reported to have enhanced antimicrobial activity, compared to an alginate sponge, against S. aureus and K. pneumonia. A microwave-assisted synthesis of alginate-stabilised Ag NPs in aqueous medium has been reported, in which sodium alginate served as both the reducing and stabilising agent. The alginate-stabilised Ag NP suspension was found to be active against E. coli and S. aureus (though the final silver concentrations of the samples tested were not reported). No attempts were made to prepare a hydrogel from the Ag NP/alginate suspension. Alginate hydrogel microbeads (˜488 μm in diameter) have been prepared which incorporate Ag NPs through the electrostatic extrusion of alginate colloid solutions containing electrochemically synthesised Ag NPs. The antimicrobial activity of the microbeads against S. aureus was demonstrated, with results indicating that up to 32 μg mL⁻¹ (˜0.3 mM) silver was released from the microbeads in the form of either Ag⁺ ions or Ag NPs, inducing the bactericidal effect.

Hydrogel networks not based on alginate have also been used for the incorporation and/or preparation of Ag NPs. For instance, Ag NPs have been prepared within hydrogel networks based on N-isopropylacrylamide and sodium acrylate through the in-situ reduction of AgNO₃ by NaBH₄. Such nanoparticle-containing hydrogels prepared through the in-situ reduction of a metal salt contained within the polymer network do not offer the opportunity to precisely tune the size and/or surface chemistry of the Ag NPs, which has implications for their subsequent antibacterial activity.

There remains a need for a simple approach for the preparation of Ag NPs of a specific size and a narrow size distribution, as well as having the ability to functionalise the surface of the particles with specific molecules so that the Ag NPs can serve as cross-linking sites within the gel, thereby enabling the incorporation of the Ag NPs into the cross-linked hydrogel matrix. The applicant has now developed a gel structure that stabilises Ag NPs through physical cross-linking, therefore preventing their aggregation or deactivation.

It is therefore an object of the invention to provide a gel comprising Ag NPs useful for treating or preventing microbial infections, or to at least provide a useful alternative.

### SUMMARY OF THE INVENTION

In a first aspect of the invention there is provided a gel comprising:
(i) nano-sized particles of metallic silver (Ag);
(ii) polymer chains comprising carboxylate groups;
(iii) carboxylate molecules; and
(iv) metal ions;
   wherein:

at least some of the Ag particles are bound to each other through a carboxylate-metal ion bridge as shown in formula (I):

Ag—X-M-X—Ag (II)

where X is a carboxylate molecule, and M is a metal ion; and at least some of the Ag particles are bound to a polymer chain through a carboxylate-metal ion bridge as shown in formula (II):

Ag—X-M-Y (II)

where X is a carboxylate molecule, M is a metal ion, and Y is a carboxylate group of the polymer chain.

In a second aspect of the invention there is provided a method of preparing the gel comprising the steps:
(i) treating a Ag salt with a reducing agent to form nano-sized particles of Ag;
(ii) treating the particles of Ag with a carboxylic acid to form a solution of Ag-carboxylate molecules;
(iii) treating the Ag-carboxylate molecules with a polymer in the presence of one of more metal ions to form the gel.

In another aspect of the invention there is provided the use of the gel for treating of preventing a microbial infection.

### BRIEF DESCRIPTION OF THE FIGURES

FIG. 1 shows a pH titration graph for an aqueous suspension of thioctic acid-coated Ag NPs.

FIG. 2 shows TEM images for thioctic acid-coated Ag NPs.

FIG. 3 is a graph showing the particle size distribution in an aqueous suspension of thioctic acid-coated Ag NPs measured from multiple TEM micrographs.

FIG. 4 shows

FIG. 5 shows Cryo-TEM micrographs of an Ag NP-containing alginate gel.

FIG. 6 shows a proposed structure of Ag NP-containing alginate gel.

FIG. 7 shows

FIG. 8 shows SEM images of untreated control biofilms for a) S. gordonii, b) S. mitis, c) S. mutan, d) S. oxford, e) E. faecalis, f) E. coli, and g) P. aeruginosa.

FIG. 9 shows SEM images of biofilms treated with alginate gel (with no Ag NPs) for a) S. gordonii, b) S. mitis, c) S. mutan, d) S. oxford, e) E. coli, and f) P. aeruginosa.

FIG. 10 shows SEM images of biofilms treated with Ag NP-containing alginate gel via the direct contact method for a) S. gordonii, b) S. mitis, c) S. mutan, d) S. oxford, e) E. faecalis, f) E. coli, and g) P. aeruginosa.

FIG. 11 shows SEM images of biofilms treated with Ag NP-containing alginate gel via the nitrocellulose membrane method for a) S. gordonii, b) S. mitis, c) S. mutan, d) S. oxford, e) E. faecalis, and f) P. aeruginosa.

FIG. 12 shows the antifungal activity of Ag NP-containing alginate gel against Candida albicans ATCC10261.

FIG. 13 shows PCA scores plot in 2-D PC space of microbiological samples at im-plant sites, no disease versus disease.

FIG. 14 shows PCA scores plot in 2-D PC space of microbiological samples at premolar sites, no disease versus disease.

### DETAILED DESCRIPTION

The gel of the invention comprises a polymer matrix containing Ag NPs. Some of the Ag NPs are bound to each other via alkylcarboxylate molecules and metal ions, whereas other Ag NPs are bound to polymer chains also via alkylcarboxylate molecules and metal ions.

The term “nano-” or “nano-sized” means having at least one size, dimension or scale in the nanometre range, typically several nanometres to several hundred nanometres. A “nanoparticle” or “NP” is therefore any particle having at least one dimension, e.g. diameter, in the range of several nanometres to several hundred nanometres.

The term “alkyl” means any hydrocarbon moiety including whether branched or straight chained and whether saturated or unsaturated.

The term “carboxylate” means the conjugate base of a carboxylic acid, i.e. —COO⁻.

The term “alkylcarboxylate” means the conjugate base of an alkylcarboxylic acid, i.e. RCOO⁻ where R is an alkyl group.

The term “gel” means a substantially dilute cross-linked system which exhibits no flow when in the steady-state.

The term “hydrogel” means a gel comprising a network of polymer chains that are hydrophilic. Hydrogels are highly absorbent (they can contain over 90% water) natural or synthetic polymeric networks.

The term “polymer” means a synthetic or natural macromolecule comprising multiple repeated subunits.

The term polymer chain” means a length of polymer comprising multiple subunits linked together in the form of a chain.

The gel of the invention comprises:
(i) nano-sized particles of metallic silver (Ag);
(ii) a polymer comprising carboxylate groups;
(iii) carboxylate molecules comprising at least one group capable of binding to Ag; and
(iv) metal ions;
   wherein:

at least some of the Ag particles are bound to each other through a carboxylate-metal ion bridge as shown in formula (I):

Ag—X-M-X—Ag (I)

where X is a carboxylate molecule, and M is a metal ion; and at least some of the Ag particles are bound to a polymer chain through a carboxylate-metal ion bridge as shown in formula (II):

Ag—X-M-Y (II)

where X is a carboxylate molecule, M is a metal ion, and Y is a carboxylate group of the polymer.

In some embodiments of the invention the polymer is a polysaccharide, for example alginic acid, hyaluronic acid, polyglutamic acid, polygalacturonic acid, and carboxymethyl cellulose or any other suitable polysaccharide or mixture of polysaccharides.

The polymer may comprise a backbone polymer chain (which may or may not be a polysaccharide) and may comprise polysaccharide chains, for example alginate or modified alginate chains as side chains or auxiliary chains from the backbone polymer chain. Further, the polysaccharide chains may be cross-linked between side chains, auxiliary chains and/or backbone chains.

In some embodiments of the invention the group capable of binding to Ag is a thiol group or an amine group.

The alkylcarboxylate molecules may comprise two or more groups capable of binding to Ag, for example the two thiol groups of a disulfide moiety.

In some embodiments of the invention, the carboxylate molecules are alkylcarboxylate molecules. Preferably, the alkylcarboxylate molecules are straight chain or branched, cyclic or acyclic, aromatic or non-aromatic C₄-C₁₀alkylcarboxylate molecules.

Examples of the carboxylate molecules include, but are not limited to, 6-mercaptohexanoic acid, 8-mercaptooctanoic acid, mercaptosuccinic acid, 4-mercaptobenzoic acid, 4-mercaptophenylacetic acid, lipoic acid (thioctic acid), dihydrolipoic acid, glutathione, penicillamine, 5-(4-amino-6-hydroxy-2-mercapto-5-pyrimidinyl)pentanoic acid, and 2-mercapto-4-methyl-5-thiazoleacetic acid.

Although the gel may comprise any suitable metal ions, or mixtures of metal ions, divalent metal ions are preferred, for example Ca²⁺, Zn²⁺ or Sr²⁺ ions.

In some embodiments of the invention, the Ag is present in the gel at a concentration in the range 230 to 1025 μg/mL.

The gel of the invention may be prepared according to any suitable method. One method comprises the steps:
(i) treating a Ag salt with a reducing agent to form nano-sized particles of Ag;
(ii) treating the particles of Ag with a carboxylic acid to form a solution of Ag-carboxylate molecules; and
(iii) treating the Ag-carboxylate molecules with a polymer in the presence of one of more metal ions to form the gel.

The Ag salt in step (i) may be present in aqueous solution, as a dispersion in water, or present in the form of a microemulsion.

The gel of the invention has been found to exhibit both antibacterial and antifungal activity. The gel is expected to be active against a wide range of bacteria including, but not limited to, Streptococcus mutans, Streptococcus mitis, Streptococcus gordonii, Enterococcus faecalis, Staphylococcus oxford, Pseudomonas aeruginosa or Escherichia coli. The gel is also expected to be active against wide range of fungi including, but not limited to, Candida albicans.

The gel may be applied topically to any site for the purpose of treating or preventing an infection.

The invention is described below in detail with reference to the stabilisation of Ag NPs using thioctic acid (lipoic acid), but it will be appreciated that various carboxylate molecules will function in the same way.

Example 1 describes the preparation of thioctic acid-stabilised Ag NPs. The aqueous suspensions of thioctic acid-coated Ag NPs at pH 9 appeared brown in colour, which is characteristic of very small dispersed Ag NPs at high concentration. Depending on the synthetic conditions used, the final silver concentration of the suspensions ranged between 230 and 1025 μg mL⁻¹. This allows for significant flexibility around the volume and concentration of the Ag NP suspension that can be incorporated into an alginate gel.

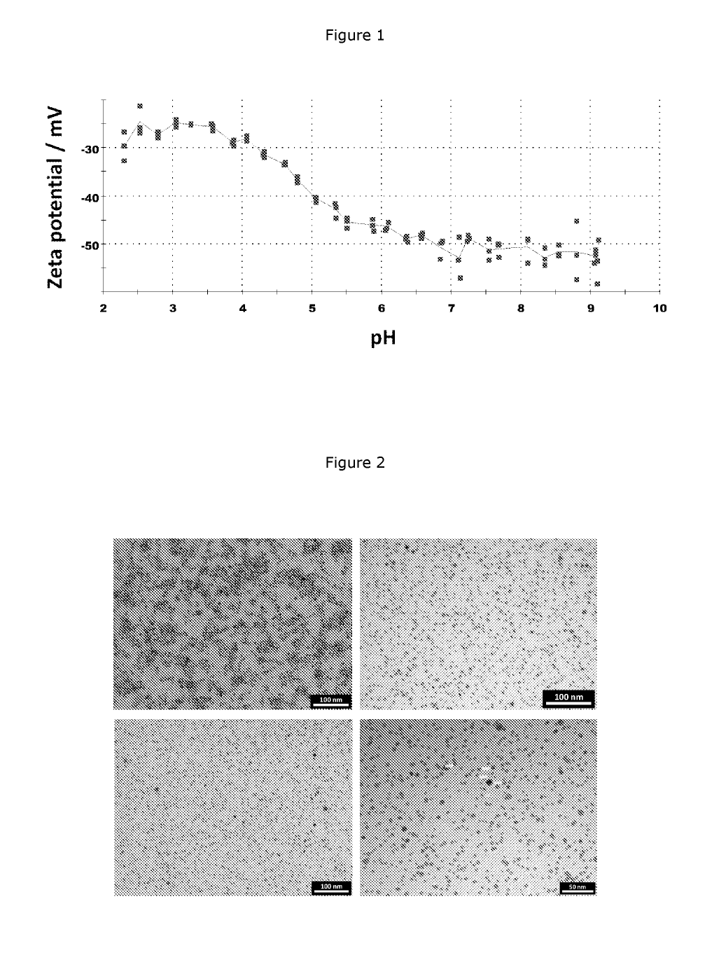

The zeta potential of samples of thioctic acid-stabilised Ag NPs was measured according to Example 2. The stability of thioctic acid-capped metal nanoparticles in water was observed to be pH dependent, as expected. FIG. 1 shows the zeta potential profile of the suspensions as a function of pH from pH 9.11-2.30, with 0.1 M HCl used as the titrant. The results show that at pH>4, the negative charge of the deprotonated carboxylic group (FIG. 1) imparts sufficient electrostatic repulsive forces between the nanoparticles in suspension for the colloid to remain stable over time, as evidenced by ζ<−30 mV. Conversely, at suitably low pH (pH<4), an increase in protonation of the carboxylic group results in ζ≥−30 mV, leading to flocculation of the nanoparticles, and destabilisation of the colloid. This pH-dependent flocculation was confirmed for thioctic acid-coated silver nanoparticles by alternating the pH of the suspension between ˜2 and ˜9 with HCl and NH₄OH, and observing a reversible flocculation and redispersion of the nanoparticles.

In order to maintain Ag NP stability in suspension over time (to ensure suitable shelf life), the suspensions may be adjusted to pH 9 immediately after preparation (ζ˜−53 mV), and stored in the dark at this pH for subsequent incorporation into alginate gels.

Transmission electron microscopy (TEM), as described in Example 3, was used to check for aggregation of Ag NPs. Representative TEM images for thioctic acid-coated Ag NPs are shown in FIG. 2. The micrographs show discrete and separated Ag NPs, with no evidence of aggregates, indicating successful and efficient coating of the NPs by thioctic acid.

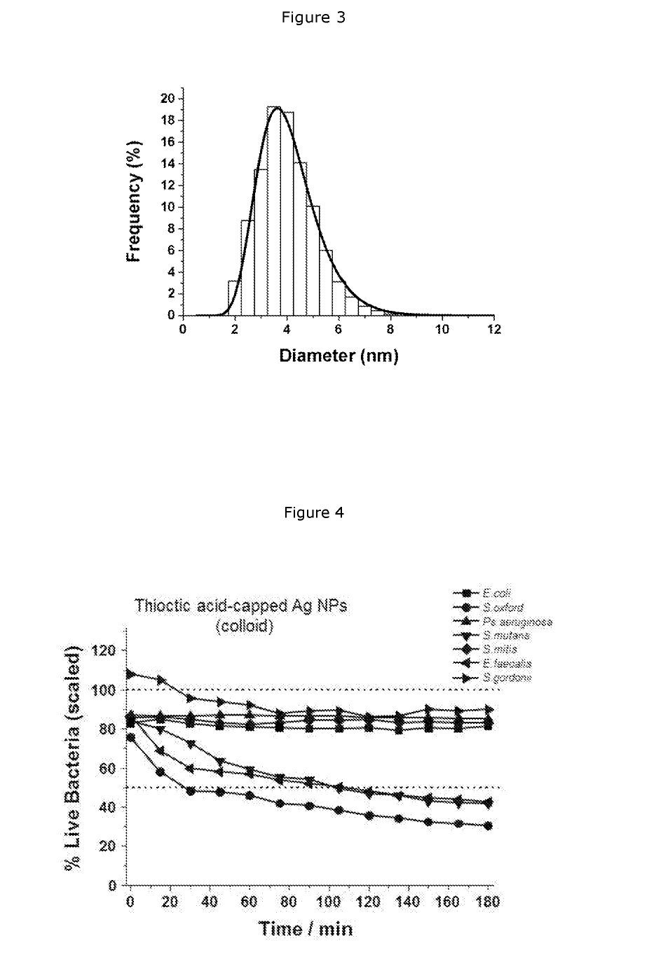

A statistical sample of the particle size was obtained by direct measurement of the diameters of 1595 particles. From these measurements, a particle size distribution histogram was prepared, which is shown in FIG. 3. The distribution was fitted successfully to a log-normal size distribution, from which the mean particle diameter and standard deviation was derived (4.1±1.6 nm). The fact that the majority of the particles are less than 7 nm in diameter indicates a reduced risk of nanotoxicity in mammals.

The antimicrobial activity of thioctic acid-stabilised Ag NPs in colloidal form was determined according to Example 4. The ratio of green to red fluorescence signals (F_{G/R}) obtained from each well in the microtitre place was calculated, and used as a measure of the relative abundance of viable bacterial cells within each well. The average F_{G/R} value was calculated from three replicate experiments for each thioctic acid-capped Ag NP treatment, for each type of bacteria, at each time point. The data was normalised by dividing the mean F_{G/R} value at each time point to that obtained for the untreated control bacteria at the same time point, and the results are reported as a percentage. Thus, the results presented reflect the change in the relative abundance of live bacteria (as a percentage) following exposure to thioctic acid-capped Ag NPs for various amounts of time, compared to the untreated cells (control samples), which have been normalised to 100%.

The antimicrobial activity of a given volume (11.6 μL) of an aqueous suspension of thioctic acid-capped Ag NPs was tested against seven microorganisms. The silver concentration of the suspension was determined by ICP-MS, according to the method of Example 5, to be 431 μg mL⁻¹. Therefore, the total mass of silver added to each well in the microtitre plate was 5.0 μg. A decrease in the percentage of live bacteria over time can be seen from FIG. 4 which displays the time dependence of the antimicrobial activity, investigated at 15 min intervals for a total of 180 min.

Alginate gels were prepared in the absence of Ag NPs to serve as a control, against which future Ag NP-containing gel formulations were to be compared. The gels were prepared using a well-established approach, as described in Example 6. A source of calcium ions was added to an aqueous solution of sodium alginate, resulting in gelation through rapid cross-linking with Ca²⁺. CaCl₂ was used as the source of Ca²⁺ ions, as is frequently reported in the literature, but the use of alternative sources have also been reported for the fabrication of alginate gels, including CaSO₄ and CaCO₃. The source of Ca²⁺ ions is often selected based on the desired rate of gelation, as gelation speed is known to affect the uniformity and strength of the resulting gel. CaSO₄ and CaCO₃ are less soluble than CaCl₂ in aqueous solution, and are therefore typically selected for use when a slower and/or more controlled gelation process is preferred. However, additional reagents may also be required for gelation to proceed. For instance, CaCO₃ is not soluble in water at neutral pH. As a consequence, D-glucono-δ-lactone (GDL) is often added to an alginate solution containing CaCO₃ in order to decrease the pH, causing the dissociation of Ca²⁺ from CaCO₃.

Ag NP-containing alginate gels were prepared according to Example 7. Ag NPs were incorporated into the alginate gel formulation described above by the addition of 0.1 M CaCl₂.2H₂O to an aqueous suspension of sodium alginate (pH 9) containing thioctic acid-capped silver nanoparticles. A coloured, malleable gel was immediately formed. The NP-containing gel was observed to have the same consistency, uniformity and injectability as the alginate-only gel. However, the most obvious immediate difference between the two formulations was the bright colour of the Ag NP-containing gel. The evenness of the colour provides strong evidence that the gel contains a uniform distribution of Ag NPs, or very small clusters of Ag NPs, throughout the matrix, as opposed to a few discrete areas containing a higher than average density of NPs, or large clusters of NPs.

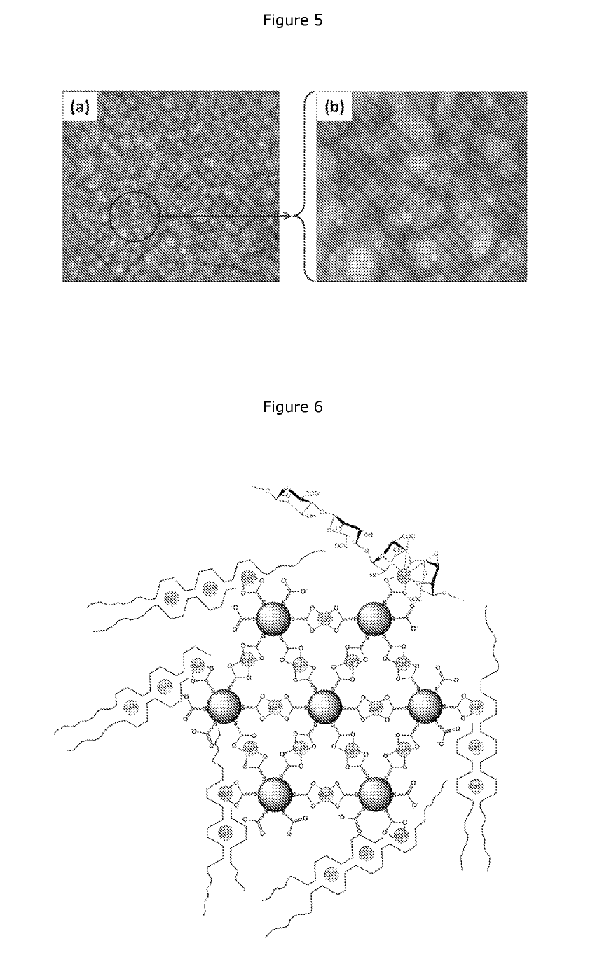

Cryo-TEM analysis was performed on the Ag NP-containing alginate gels in order to obtain high resolution images of the interpenetrating membrane network and heterogeneous morphology of the alginate hydrogel, as well as to obtain information about the distribution of thioctic acid-capped Ag NPs throughout the gel matrix. A representative Cryo-TEM image of the Ag NP-containing gel is shown in FIG. 5(a). The porous structure of the gel can be clearly seen. The interconnected gel network appears dark, while the surrounding/encapsulated vitreous water appears light. An expanded view of a randomly-selected, small area of the micrograph is shown in FIG. 5(b). This image reveals a high density of small assemblies of Ag NPs, of fairly uniform size, distributed throughout the gel matrix, as intended. There appears to be uniform inter-particle spacing between the discrete Ag NPs composing the assemblies, with the distance between neighbouring Ag NPs less than the diameter of a single NP. This observation indicates successful and persistent ionic (Ca²⁺) cross-linking between the Ag NPs forming the assemblies. This is favourable as inter-particle spacing signifies maximum exposure/availability of the Ag NP surface, which is likely to be critical for achieving optimum antimicrobial activity.

Based on the results obtained for the Ag NP-containing gel, a proposed structure of the gel is shown schematically in FIG. 6. In this model structure, the Ca²⁺ ions have three roles. First, they can be seen linking thioctic acid-capped Ag NPs together into small assemblies through NP-Ca²⁺-NP bridging, utilising the terminal carboxylate groups of the thioctic acid molecules at the surface of the Ag NPs. Second, the Ca²⁺ ions can be seen bridging the Ag NP assemblies to alginate polymer strands through NP-Ca²⁺-L-guluronate ionic cross-links, again utilising the terminal carboxylate groups of the thioctic acid molecules. The fact that alginate surrounds and stabilises the small Ag NP assemblies was confirmed by Cryo-TEM analysis (FIG. 5(a)). Finally, the Ca²⁺ ions are shown linking G-blocks of adjacent alginate chains through interactions with the carboxylate moieties.

The antimicrobial activity of 0.1 g of Ag NP-containing gel (total silver content of 8.9 μg, as determined by ICP-MS) was tested against seven microorganisms according to Example 4. The gel had an immediate effect on the population of live bacteria. Upon exposing each type of bacteria to the gel (t=0), there was a rapid decrease in the number of live bacteria for all organisms except Ps. aeruginosa. Of the seven types of organisms tested, five showed a greater decrease in the percentage of live bacteria at time t=0 than was observed for the aqueous suspension of thioctic acid-stabilised Ag NPs at an identical silver concentration. Furthermore, all seven types of organisms tested showed a greater decrease in viable bacteria at time t=0 than was observed for the same mass of alginate-only gel, thus confirming the antimicrobial activity was conferred by the Ag NPs within the gel.

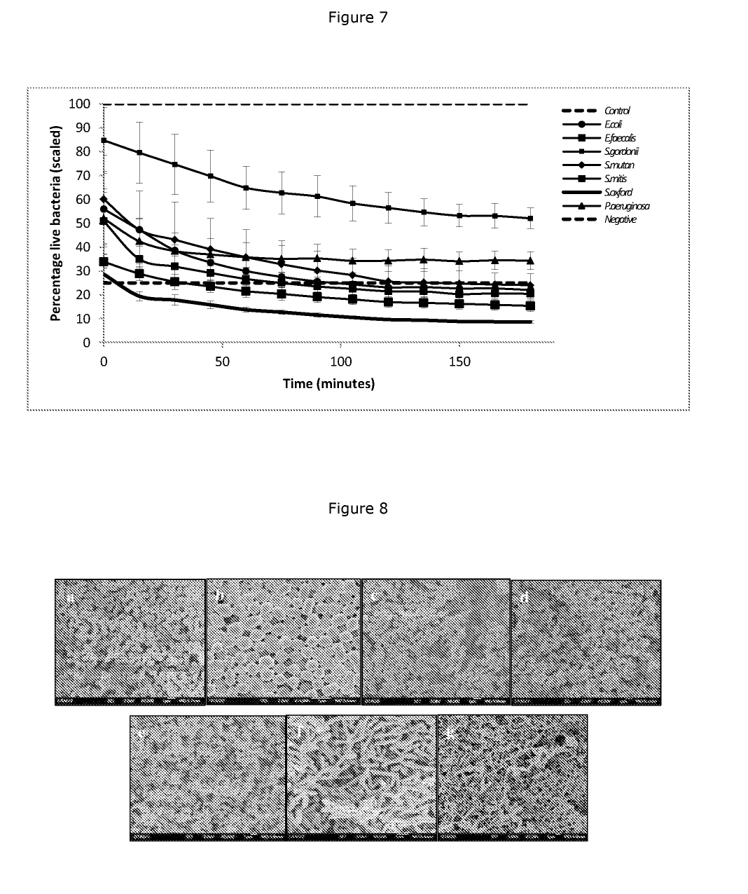

The bacterial population was monitored by fluorescence measurements performed every 15 min over a 3 hour time period, and the results are shown in FIG. 7. Notably, all organisms (with perhaps the exception of S. mutans) showed a time-dependent decrease in the number of live bacteria which continued throughout the entire 180 min experiment.

The treatment of biofilms is an important phase within the development and evaluation of an antibacterial medicament. Biofilms are densely packed communities of adherent, surface-bound bacteria surrounded by a matrix of extracellular polymeric substance (EPS). This organised bacterial community forms a higher level of structure than free-floating planktonic cells, and undergoes gradual stages of cell adherence, proliferation and maturation, providing rigidity to the complex architecture. There are both physiological and behavioural differences between microorganism contained within a biofilm and their planktonic cell counterparts. Because of this, biofilms often demonstrate reduced susceptibility to antibiotic treatment. Thus, in order to assess the antimicrobial activity of a particular therapeutic on clinically-relevant biofilms, it is not appropriate to simply extrapolate results obtained from experiments performed using planktonic cells.

Bacterial biofilms were prepared according to Example 8. Example 9 describes an assay for the susceptibility of bacterial biofilms. SEM analyses were conducted according to Example 10 and confocal laser scanning microscopy (CLSM) analyses were performed according to Example 11. Representative SEM images obtained for untreated (control) biofilms are shown in FIG. 8. The seven images show biofilms formed from each of the seven types of organisms investigated. They show that the well-defined cell morphologies known to be typical of cocci and bacilli bacteria were obtained in the control biofilm samples. The cells appeared as individual healthy cells in robust biofilm form. The cell morphologies were as expected. Specific features characteristic of biofilms can be identified in the images, such as a high density of cells in a small area, and the presence of EPS, which appear as ‘stringy connections’ between cells. EPS is a feature of strong, well-built and resistant biofilms.

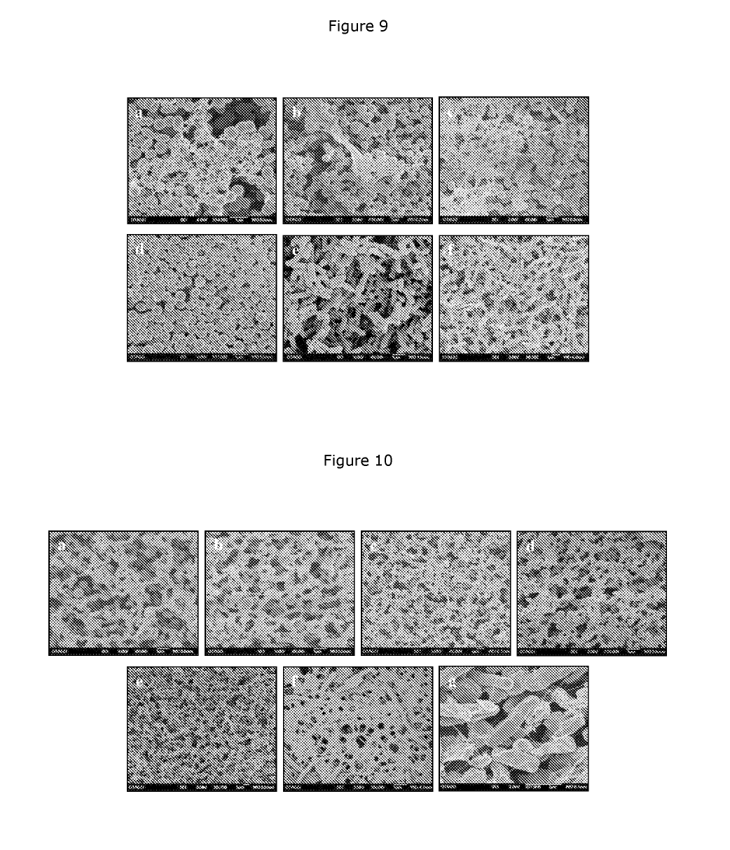

Representative SEM images obtained for biofilms treated with an alginate gel (with no Ag NPs) are shown in FIG. 9. The six images show treated biofilms that were formed from each of six types of microorganisms investigated. Alginate gel-treated biofilms were observed to exhibit enhanced EPS production. EPS appeared more abundant in the SEM images of the treated biofilms all cases. The well-defined cell morphology was retained in all cases, indicating that the bacteria remained healthy following alginate gel treatment.

Representative SEM images obtained for biofilms treated with Ag NP-containing alginate gel via direct contact are shown in FIG. 10. The seven images show treated biofilms that were formed from each of the seven types of organisms investigated. They show considerable morphological changes to the cells (when compared to the controls), and the development of cellular aggregates and irregular cellular shapes. When the Ag NP-containing alginate gel was applied directly to the biofilm, in addition to irregular cellular morphology, a high number of cellular aggregates was observed in the SEM images, and in some cases, obviously flattened surfaces were observed. These features are consistent with cell death. Furthermore, in some cases there appears to be a formation of an interconnected network of aggregated material, presumably composed of intracellular material from the lysed bacteria, giving an appearance of “melted cells”. This is particularly apparent in the SEM images obtained for E. faecalis (FIG. 10(e)) and E. coli (FIG. 10(f)). The bacterial cells of P. aeruginosa biofilms treated with Ag NP-containing alginate gel (FIG. 10(g)) have a collapsed or “deflated”-type appearance, likely due to dehydration effects.

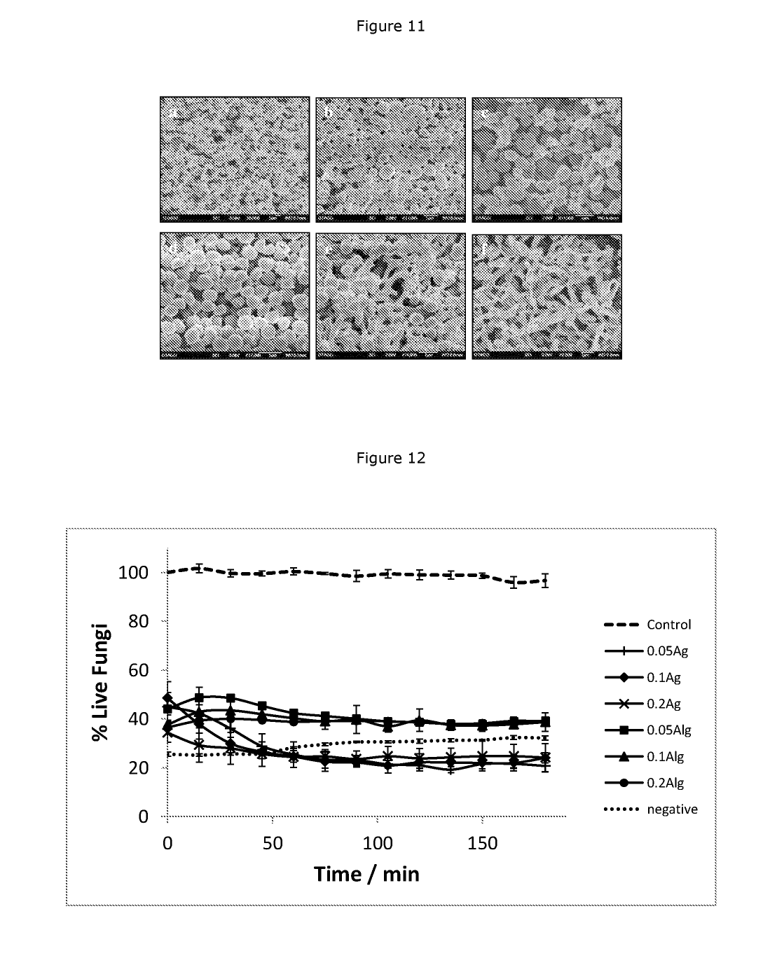

Representative SEM images obtained for biofilms treated with Ag NP-containing alginate gel via the nitrocellulose membrane method are shown in FIG. 11. When the Ag NP-containing alginate gel is applied on top of a nitrocellulose membrane, the same changes to the bacterial cells appear to occur as when the gel is applied via direct contact (i.e. significant disruption to cell morphology), although not quite as pronounced.

The percentage of red versus green fluorescence that was detected throughout the total biomass of both treated and untreated biofilms by CLSM analysis, as described in Example 11, is reported in Table 1. From these results, it is clear that in all cases, treatment of the biofilms with Ag NP-containing alginate gel results in a significant increase in the amount of red fluorescence, compared with the untreated controls, which represents a large increase in the number non-viable cells within the biofilm.

TABLE 1
Percentage of the total biomass (within the
total biovolume) that fluoresces red or green

| | |
|---|---|
| Control | Ag NP-gel treated |

| | green | red | green | red |
|---|---|---|---|---|
| S. gordonii | 99 | 1 | 53 | 47 |
| S. mitis | 77 | 23 | 56 | 44 |
| S. mutan | 96 | 4 | 49 | 51 |
| S. oxford | 72 | 28 | 56 | 44 |
| E. faecalis | 72 | 28 | 43 | 57 |
| E. coli | 71 | 29 | 39 | 61 |
| P. aeruginosa | 69 | 31 | 41 | 59 |

Example 12 describes an assay to assess the penetration of silver, in either ionic or NP form, through the biofilm to reach the agar below. Inductively coupled plasma-mass spectrometry (ICP-MS) analysis of the agar was performed to quantify the mass of silver contained within a known mass of agar. This assay was performed on a biofilm composed of E. coli bacterial cells. ICP-MS analysis of the agar sample removed from the area below the treated biofilm (and polycarbonate membrane) revealed that 0.58 □g of silver was contained within 0.05 g of the agar (6.3% of the total amount of silver administered through the colloidal treatment). A similar analysis performed on untreated biofilms revealed that <0.00005 □g of silver was contained within 0.05 g of the agar (i.e., if there was any silver present at all, it was below the detection limit of the instrument). These results undoubtedly demonstrate the successful penetration of a portion of the silver through the entire mass of the biofilm to reach the agar below.

The Ag NP-containing alginate gel of the invention was also found to have antifungal properties. Example 13 shows that the gel has a strong fungicidal effect. An alginate gel (0.5, 0.1 and 0.2 g) containing a mix of Ca²⁺, Zn²⁺ and Sr²⁺ ions, but not containing Ag NPs, was found to significantly decrease the population of viable fungi, but not below the level of the negative control. However, the alginate gel in the same amounts (0.5, 0.1 and 0.2 g) containing the same mix of metal ions and Ag NPs (even at very low Ag NP concentration) was found to decrease the viable fungi population below the level of the negative control. The results are shown in FIG. 14.

Validation of the antibacterial activity and biocompatibility of the gel of the invention for the treatment and prevention of peri-implantitis and periodontal disease in humans was performed in a novel sheep model with a split-mouth design, consisting of artificial ligature-induced periodontitis around teeth and (on the contralateral side) peri-implantitis around a pair of dental implants, one with a blasted surface and the other with a oxidised surface. Disease lesions were established in sheep and the antimicrobial gel formulation applied. Sacrifice of the sheep allowed histomorphometric analysis of the periodontal and peri-im plant tissues, to confirm the efficacy of antimicrobial activity at preventing disease progression, facilitating subsequent potential strategies to regenerate lost tissue.

The high throughput sequencing of microbiology studies of Example 14 gave an indication of the various bacterial genera present within the samples. All genera identified through sequencing are known to be found within the oral microbiome. However, the initiation of periodontitis/peri-implantitis is dependent on a multitude of factors. One of these factors is the particular level/ratio of various genera, which could increase the possibility of the occurrence of a pathogenic infection. The samples obtained from animals prior to ligature placement, termed ‘baseline’ samples (taken from both the implant and premolar sites), when compared to the diseased samples, consisted of a higher abundance of proteobacteria (implant site p<0.00003), which are Gram-negative bacteria commonly found within the environment and within the oral cavity. Notably, the bacteroidetes, such as Prevotella, Bacteroides, Porphyromonas, and Tannerella, were consistently detected at higher levels in diseased sheep samples compared to baseline samples (implant site p<0.01), which is consistent with literature reports related to periodontitis and peri-implantitis. The remaining genera identified in the samples, Fusobacteria, Firmicutes, Actinobacteria, and SR1, are Gram-positive anaerobes that are known to prominently feature in periodontal infection sites. Synergistetes are not encountered in subgingival plaque from periodontal healthy gums. Spirochaetes are coiled cells that are found in root canal infections, pericoronitis, gingivitis and periodontitis, and have been reported to constitute up to 56% of the flora in advanced marginal periodontitis. All genera, apart from proteobacteria, appeared to increase in abundance when periodontal disease was induced for both implant and premolar sites (Implant sites: Fusbacteria p<0.04, Firmicutes p<0.01, SR1 p<0.003, Synergistetes p<0.0003 and Spirochaetes p<0.06, a statistical difference was not indicated by Actinobacteria). The statistics were conducted using an F-test to indicate variance between the two baseline and diseased sample groups and an unequal or equal two-tailed t-test dependent on the data.

Microbiological data suggests that this ligature-induced model of periodontal and peri-implant disease was populated by bacterial flora consistent with periodontal and peri-implant disease and distinctly different from the flora associated with healthy oral sites.

The histology studies of Example 14 indicate that the Ag gel of the invention had an effect in reducing inflammation and promoting healing around teeth and implants, and that this effect persisted for up to 4 months (equivalent to 5-6 months in humans).

After one week, premolar teeth in the test animals (N=2 sheep) showed excellent healing. There were some remnants of material that may have been Ag gel. The healing in the one week test animals seemed better than the control animals (N=2), which showed a more pronounced inflammatory infiltrate and persisting periodontal pocket. The anterior implants (both test and control) had much greater inflammatory reaction than the posterior implants, suggesting that the type of implant surface may have had an influence on the effectiveness of the gel treatment. One of the control sheep had already lost one Nobel implant prior to creation of the surgical defect. There was little to distinguish between the test and control one-week implants in the anterior position (N=2 sheep per group). The test one-week posterior implants (Ag-gel treated Southern Implants) appeared to have a smaller inflammatory infiltrate that was confined to the most apical part of the surgically-created chronically-inflamed defect, whereas the control implants (scaling-only, Southern Implants), although better integrated than the anterior (Nobel) implants, had a larger inflammatory infiltrate that extended into the marrow spaces and trabecular bone at the base of the surgical defect. These results suggest that a single application of the gel formulation to the posterior Southern implants was effective in reducing inflammation.

After 16 weeks, the premolar teeth in the test animals (N=2 sheep) showed good healing with little sign of epithelial down-growth and only minor inflammatory infiltrate. Bone had regenerated coronal to the surgical defect. Premolar teeth from the control animals (N=2 sheep) had more marked, persisting inflammation with little sign of bone regeneration and deeper pockets. These results suggest that a single application of the gel treatment had effects on inflammation in a ligature-induced artificial periodontitis model, that persisted for up to 4 months after application.

For the 16 week implants, only one Nobel implant in the anterior position remained. However, the implant was well integrated and the surgically-created peri-implantitis lesion appeared to show little sign of inflammation or progression. Both posterior implants in the two test sheep were still present and well integrated with evidence of only limited inflammation and some bony repair. By comparison, the two control sheep had only one surviving anterior implant and no surviving posterior implants between them, and the sole remaining anterior implant had completely lost osseointegration, was surrounded by a large inflammatory infiltrate and would shortly have been lost as well. This suggests that the single application of the gel was effective in limiting or reducing progressive inflammation and bone loss in a ligature-induced artificial animal model of severe peri-implantitis, and that this effect persisted for up to 4 months after a single application.

Any reference to prior art documents in this specification is not to be considered an admission that such prior art is widely known or forms part of the common general knowledge in the field.

As used in this specification, the words “comprises”, “comprising”, and similar words, are not to be interpreted in an exclusive or exhaustive sense. In other words, they are intended to mean “including, but not limited to.

The invention is further described with reference to the following examples. It will be appreciated that the invention as claimed is not intended to be limited in any way by these examples.

### EXAMPLES

Materials and methods

Sodium bis(2-ethylhexyl)sulfosuccinate (AOT, 99%), silver nitrate (AgNO₃, 99%), 1,2-dithiolane-3-pentanoic acid (thioctic acid, 99%), sodium borohydride (NaBH₄, 98%) and sodium alginate (low viscosity) were purchased from Sigma-Aldrich. Calcium chloride dihydrate (CaCl₂.2H₂O, >99%) was purchased from Global Science and n-heptane (95%) was purchased from Univar. Deionised (DI) water was purified by a Millipore Milli-Q RG ultra-pure water system. A LIVE/DEAD® BacLight™ Bacterial Viability Kit (L7012) was purchased from Life Technologies. The kit contained two fluorescent dyes used as viability probes: SYTO® 9 (3.34 mM in DMSO) and propidium iodide (PI, 20 mM in DMSO).

### Example 1

### Preparation of Thioctic Acid-Stabilised Ag NPs

Two microemulsions were prepared. The first microemulsion was prepared by adding an aqueous solution of AgNO₃ (0.13 M, ω=10) to a mixture of AOT (0.33 M) in n-heptane, and the second microemulsion was prepared by adding an aqueous solution of NaBH₄ (1.84 M, ω=10) to AOT in n-heptane (0.33 M). The microemulsion containing NaBH₄ was added drop-wise with stirring to the microemulsion containing AgNO₃. A rapid colour change from light yellow to a dark yellow-brown colour was observed. The mixture was allowed to stir in the dark for 5 h, after which 1 mM thioctic acid (dissolved in 0.25 mL ethanol) was added to the Ag NP-containing μ-Em system, and the mixture was left to stir for an additional 1 h. Stirring was discontinued, and a volume of 1:1 acetone/methanol was added to the mixture (with the volume equivalent to the total volume of n-heptane used), releasing the thioctic acid-coated Ag NPs from the AOT RMs, and two distinct phases immediately became visible; a brown coloured, non-polar organic ‘upper’ phase, and a cloudy-white-coloured polar ‘lower’ phase. The upper phase became colourless when the reaction vessel was allowed to sit undisturbed overnight, and dark-coloured particles were then observed at the liquid-liquid interface. The organic phase was removed from the system and discarded. Particles from the interface were collected, washed 3 times with ethanol, then redispersed with mixing in 1 to 6 mL of DI water, as required, with the pH of the water pre-adjusted to pH 9 with NH₄OH. The resulting yellow-brown coloured aqueous suspension was then centrifuged twice at 13,000 rpm for 45 min, and the supernatant was set aside for further characterisation.

### Example 2

### Zeta Potential

Electrophoretic mobility of thioctic acid-stabilised Ag NP samples were measured at 25° C. on a Malvern Zetasizer (Malvern Instruments; Malvern, UK) using the M3-PALS technology.⁵⁵ The zeta potential of the samples was calculated by means of the Smoluchowski equation using Zetasizer software (v.6.20; Malvern Instruments). An automated pH titration was also performed in which zeta potential measurements were performed over a pH range of 9.11-2.30 using the MPT-2 autotitrator accessory (Malvern Instruments; Malvern, UK). 0.1 M HCl was used as the titrant. The results are shown in FIG. 1.

### Example 3

### Transmission Electron Microscopy

Transmission electron microscopy (TEM) images were obtained using a Philips CM100 BioTWIN transmission electron microscope (Philips/FEI Corporation; Eindhoven, Holland) equipped with a LaB6 emitter fitted with a MegaView III Olympus digital camera. Samples were prepared for analysis by depositing a volume of 10 μL onto carbon-coated (400-mesh) copper grids. After 60 sec, the excess volume was carefully blotted with filter paper and the sample was allowed to air dry before analysis. The nanoparticle size was determined by a particle detection process (a minimum of 200 particles was measured) with the analySIS 3.1 software (Soft Imaging System GmbH) using magnified TEM images.

### Example 4

### Antibacterial Activity of Thioctic Acid-Stabilised Ag NPs

Pure stock cultures of Streptococcus mutans (UAB159), Streptococcus mitis (ILB), Streptococcus gordonii (DL1), Enterococcus faecalis (JH22), Staphylococcus oxford, Pseudomonas aeruginosa (OTIS) and Escherichia coli (DH5α) were obtained from the Department of Oral Sciences, University of Otago, New Zealand. Colonies of S. mutans, S. mitis, S. gordonii, E. faecalis, S. oxford, Ps. aeruginosa and E. coli were aerobically grown in tryptic soy broth at 37° C. for 24 hours.

Bacteria preparation. 10 mL of each of the bacterial cultures were centrifuged at 7,000×g for 5 min. The supernatant was removed and the pellet was resuspended in ˜5 mL of 10 mM Tris-buffer saline (prepared with 8.5 mg mL⁻¹ NaCl and 0.1% peptone, then adjusted to pH 7.5 using 1 M HCl). The suspensions were re-centrifuged 2 more times at 5,000×g for 2 min, each time the pellet was resuspended in ˜5 mL Tris buffer. The final volume of Tris-buffered saline (pH 7.4) used for the resuspension was adjusted to obtain an optical density at 670 nm (OD₆₇₀) of 1.0, as measured by an Ultrospec 6300 pro UV-Vis spectrophotometer; Amersham Biosciences).

Preparation of live/dead stain. Stock solutions of the BacLight™ dyes were prepared as follows: Equal volumes (6 μL) of SYTO® 9 and PI were combined, mixed thoroughly, and diluted to 2 mL with sterilised DI H₂O.

Determination of bacterial viability following exposure to thioctic acid-stabilised Ag NPs. 80 μL of the bacterial preparations were placed into each of 3 wells (i.e., performed in triplicate) of a 96-well microtitre plate, followed by 100 μL of the pre-prepared dye mixture. To serve as an eventual positive control, 80 μL of ‘live’ bacterial standard were also placed into each of 3 wells, followed by 100 μL of the dye mixture. To serve as an eventual negative control, 80 μL of ‘dead’ bacterial standard (previously placed in a water bath, 70° C., 30 min) were placed into each of 3 wells, followed by 100 μL of the dye mixture. To serve as an assay control, 80 μL of Tris buffer were placed into each of 3 wells, followed by 100 μL of the dye mixture. The microtitre plate was incubated in the dark at room temperature for 15 min. 20 μL of sterilised DI H₂O were added to each of the wells selected to serve as positive and negative controls. To the remaining wells, the volume of the Ag NP suspension to be tested (6.1, 11.6 or 23.2 μL) was added to each well. The fluorescence emission from both the live (green fluorescent) and dead (red fluorescent) cells was measured at 24-25.6° C. every 15 min from t=0 to t=180 min with a Synergy 2 microplate reader (BioTek®; Winooski, Vt., USA); λ_{excitation}=485 nm; λₑₘᵢₛₛᵢₒₙ (green)=530 nm for the live stain (SYTO 9), λₑₘᵢₛₛᵢₒₙ (red)=630 nm for the dead stain (PI).

The data obtained for each bacterial suspension in each well was analysed by dividing the fluorescence intensity of the stained cell suspension (F_{cell}) at emission 1 (green) by the fluorescence intensity at emission 2 (red) to obtain the ratio of green to red fluorescence intensity (Ratio_{G/R}, or F_{G/R}), which is proportional to the relative number of live bacteria.

$${Ratio}_{G / R} = \frac{{F}_{cell , em 1}}{{F}_{cell , em 2}}$$

The triplicate measurements were used to calculate average F_{G/R} values for each of the bacterial preparations. The average F_{G/R} value obtained for each ‘live’ bacterial standard was considered to represent 100% viable bacteria. Thus, average F_{G/R} values obtained for each bacterial preparation exposed to thioctic acid-stabilised Ag NPs was divided by the average value obtained for the relevant ‘live’ standard (positive control) in order to calculate the fraction of live bacteria remaining (typically reported as a percentage).

Determination of bacterial viability following exposure to Ag NP-containing alginate gels. 0.05, 0.1 or 0.2 g of the Ag NP-containing gel to be tested was placed into each of 3 wells (i.e., performed in triplicate) of a 96-well microtitre plate, followed by 100 μL of the pre-prepared dye mixture and 100 μL live bacteria. To serve as an alginate gel control, 0.05 and 0.1 g of an alginate gel prepared with CaCl₂ was also placed into each of 3 wells, followed by 100 μL of the pre-prepared dye mixture and 100 μL live bacteria. The fluorescence emission was measured at 24-25.6° C. every 15 min from t=0 to t=180 min with a Synergy 2 microplate reader (BioTek®; Winooski, Vt., USA); λ_{excitation}=485 nm; λₑₘᵢₛₛᵢₒₙ (green)=530 nm λₑₘᵢₛₛᵢₒₙ (red)=630 nm.

### Example 5

### Determination of Silver Content

0.1 mL of each Ag NP-containing sample was prepared for inductively coupled plasma-mass spectrometry (ICP-MS) analysis through the addition of 4 mL of concentrated HNO₃ in a Teflon digestion vessel, followed by gentle heating. The samples were then digested at 95° C. for 1 h. During this time, the volume of the digested solution was reduced to around 0.2 mL, then made up to 3 mL total volume with Milli-Q water. ICP-MS analysis was performed on an Agilent 7500ce instrument (Agilent Technologies; CA, USA) to determine the silver concentration in each of the samples.

### Example 6

### Preparation of Alginate Gels

0.5 mL of 0.1 M CaCl₂.2H₂O was added drop-wise with gentle stirring to 3 mL of a 1.5% w/v aqueous sodium alginate solution (at neutral pH), resulting in instantaneous gelation.

### Example 7

### Preparation of Ag NP-Containing Alginate Gels

Sodium alginate was hydrated with 3 mL (1.5% w/v) of an aqueous suspension of thioctic acid-capped Ag NPs at pH 9 (the Ag NP concentration in the aqueous suspension was varied, depending on the desired NP density within the gel). 0.5 mL of 0.1 M CaCl₂.2H₂O was added drop-wise with gentle stirring, immediately forming a coloured gel. The colour of the gel ranged from yellow to brown, with darker colours caused by a higher Ag NP concentration. The gel may be freeze dried, and the resulting solid crushed to small granules. The hydrogel reforms and swells on reconstitution with water.

### Example 8

### Bacterial Biofilm Preparation

Biofilms were formed using a static colony biofilm assay technique. Inoculated tryptic soy broth (TSB), or brain heart infusion broth (BHI) for streptococci, were cultured overnight at 37° C. A bacterial culture with an optical density at 600 nm (OD₆₀₀) of 1.0, attributing to a 1×109 cfu mL-1 count, was used as a seeding culture. A 25 mm black polycarbonate membrane with a 0.45 μm pore size was placed onto tryptic soy agar (TSA), or Columbian sheep blood agar (CSA) for anaerobes, and 20 μL of the bacterial culture was placed onto the centre of the filter. This volume was used to produce a biofilm with a 12 mm diameter. The agar plates were inverted and incubated for 48 hours at 37° C., during which time the nutrient agar was replenished every 24 h.

### Example 9

### Bacterial Biofilm Susceptibility Assay

Following the 48 h incubation period, a biofilm susceptibility assay was performed on the mature biofilms. A schematic representation of the assay is presented in FIG. 1. The assay was performed by administering a given treatment (Table 2), and allowing the treatment to remain in place for 24 h. The treatments were either applied to the biofilm through direct physical contact by placing the treatment directly on top of the mature biofilm, or through the use of a diffusion mechanism by placing the treatment on top of a nitrocellulose membrane (12 mm in diameter; 0.45 μm pore size), as represented by FIG. 1(d). Following the 24 h treatment regime, the treatment and the nitrocellulose membrane (if present) were removed. The biofilm and polycarbonate membrane were subsequently removed from the agar, and gently rinsed with phosphate buffered saline (PBS) to remove non-adherent cells. The treated biofilm was then analysed using scanning electron microscopy (SEM) and confocal laser scanning microscopy (CLSM) techniques.

TABLE 2
Treatment methods

| Type of Treatment | Mass of Ag/μg | Application Method |
|---|---|---|
| None | N/A | N/A |
| Alginate gel | 0 | Nitrocellulose membrane |
| Alginate gel + Ag NPs | 94 | Nitrocellulose membrane |
| Alginate gel + Ag NPs | 94 | Direct contact |
| AgNO₃ solution | 701 | Nitrocellulose membrane |

### Example 10

### Scanning Electron Microscopy (SEM) Analysis

Both treated and untreated mature biofilm specimens were fixed in 2.5% glutaraldehyde in 0.1 M cacodylate buffer for a minimum of 2 h. Specimens underwent chemical dehydration using a graded series of ethanol solutions (30-100%), by immersing them in each solution for 5 min. Subsequently, critical point drying (CPD) was performed, the details of which are as follows. The specimens were immersed in 100% ethanol in the CPD chamber, and ethanol was slowly removed; CO₂ was used to replace the volume. This was conducted below 10° C., and then the temperature was increased to 35° C. Fixed specimens were placed onto aluminium SEM stubs and mounted with carbon tape. Mounted specimens were sputter coated with gold and analysed using a JEOL 6700 FESEM.

### Example 11

### Confocal Laser Scanning Microscopy (CLSM) Analysis

Stock solutions of the BacLight™ live/dead stains were prepared as follows: Equal volumes (6 μL) of SYTO® 9 and propidium iodide (PI) were combined, mixed thoroughly, and diluted to 2 mL with sterilised DI H₂O. The treated biofilms were then stained with 200 μL of these solutions and allowed to remain undisturbed in the dark for 15 min. The resulting stained biofilms were rinsed with PBS then placed on a microscope slide with a coverslip on top; excess liquid was wicked away with tissue. CLSM was performed using a Zeiss LSM 710 confocal microscope. CLSM biofilm image stacks obtained for both treated and untreated biofilms were analysed using a computer program (Heydorn, A., et al., Quantification of biofilm structures by the novel computer program COMSTAT, Microbiology, 2000, 146, 2395-2407) in order to quantify the percentage of green/red fluorescence that appears within the total biomass of each biofilm.

### Example 12

### Bacterial Biofilm Penetration Assay

Following the 48 h biofilm formation, a biofilm penetration assay was performed on the mature biofilms. The assay was performed by administering the treatment and allowing the treatment to remain in place for 24 h. In this assay, only one treatment was tested, which was an aqueous suspension of thioctic acid-coated Ag NPs, with a silver concentration of 310 μg mL⁻¹. A volume of 30 μL of the suspension was applied on top of the nitrocellulose membrane. As a result, a total mass of 9.3 μg Ag was administered in this treatment regime. Care was taken to ensure that the administered volume of the Ag NP suspension diffused solely through the membrane and not around it. After 24 h, a 10 mm diameter borer was used to remove a 0.05 g agar sample from the area directly beneath the biofilm and polycarbonate membrane, which was subsequently analysed using inductively coupled plasma-mass spectrometry (ICP-MS) to quantify the mass of silver that was contained within the agar (assumed to be the quantity that penetrated through the biofilm and membranes to reach the agar).

### Example 13

### Antifungal Activity of Gel Containing Thioctic Acid-Stabilised Ag NPs

An alginate gel containing an equal mixture of 3 types of divalent cations, Ca²⁺, Zn²⁺ and Sr²⁺, with and without Ag NPs, was prepared according to Example 7 but Instead of adding 0.5 mL of 0.1 M CaCl₂ dropwise with gentle stirring to form the gel, 0.167 mL of each of the 3 solutions were used: 0.1 M ZnCl₂, 0.1 M CaCl₂ and 0.1 M SrCl₂. The solutions were added dropwise in an alternating pattern until the total volume was used (i.e., 1 drop ZnCl₂, 1 drop CaCl₂, 1 drop SrCl₂, repeat cycle). The gels were tested against Candida albicans ATCC10261 (a type of fungus often found on implantable medical devices). The results are shown in FIG. 12. The experiment was performed according to Example 4. Briefly, a pure stock culture of Candida albicans was obtained from the Department of Oral Sciences, University of Otago, New Zealand. 10 ml of bacterial culture was grown aerobically in yeast extract peptone dextrose at 31° C. for 24 hours. The culture was centrifuged at 7,000×g for 5 min, the supernatant was removed and the pellet was resuspended in ˜5 mL of 10 mM Tris-buffer saline (prepared with 8.5 mg mL⁻¹ NaCl and 0.1% peptone, then adjusted to pH 7.5 using 1 M HCl). The suspension was re-centrifuged 2 more times at 5,000×g for 2 min, each time the pellet was resuspended in ˜5 mL Tris buffer. The final volume of Tris-buffered saline (pH 7.4) used for the resuspension was adjusted to obtain an optical density at 670 nm (OD₆₇₀) of 1.0, as measured by an Ultrospec 6300 pro UV-Vis spectrophotometer; Amersham Biosciences).

The live/dead fluorometric viability assay was utilised, combining equal volumes (6 μL) of SYTO® 9 and PI which were mixed thoroughly, and diluted to 2 mL with sterilised DI H₂O. Both the Ag NP containing gel and the gel containing no Ag NPs were syringed into wells at various weights, including 0.05 g, 0.1 g and 0.2 g, and were performed in triplicate. 100 μL of the premixed live/dead dye mixture was placed into each well. To serve as a positive control, 100 μL of C. albicans suspension was placed into each of three wells with no added gel (with or without Ag NPs). To serve as a negative control, 100 μL of ‘dead’ C. albicans (previously placed in a water bath, 70° C., 30 min) was placed into each of 3 wells, followed by 100 μL of the dye mixture. Finally, the C. albicans preparation was placed into each gel containing well (performed in triplicate for each weight) of a 96-well microtitre plate. The fluorescence was monitored immediately. The fluorescence emission from both the live (green fluorescent) and dead (red fluorescent) cells was measured at 24-25.6° C. every 15 min from t=0 to t=180 min with a Synergy 2 microplate reader (BioTek®; Winooski, Vt., USA); λ_{excitation}=485 nm; λₑₘᵢₛₛᵢₒₙ (green)=530 nm for the live stain (SYTO 9), λₑₘᵢₛₛᵢₒₙ (red)=630 nm for the dead stain (PI). The data obtained was analysed according to Example 4.

### Example 14

### Efficacy of Gel Formulation for Treating Periodontitis and Peri-Implantitis in Sheep Model

Formulation of Ag NP gel: Sodium alginate was hydrated with 10 mL (1.5% w/v) of an aqueous suspension of thioctic acid-capped Ag NPs (prepared according to Example 1) at pH 9. Equal volumes of 0.1 M CaCl₂.2H₂O, ZnCl₂ and SrCl.6H₂O (each at 0.250 mL) were added drop-wise with gentle stirring. The quantities of formed gel (-10 mL) were placed into a 250 mL round bottom flask, frozen using liquid nitrogen, and subsequently lyophilised for 24 h at room temperature at a pressure of 8.6×10⁻² mbar. The lyophilised solid was crushed until it resembled small granules. The lyophilised gel (0.03 g) was placed into 1 mL of 7% Methocel™ (DOW chemical company, methylcellulose and hydroxypropyl methylcellulose polymers) to produce a mucoadhesive composite gel containing AgNPs. The AgNP-containing gel used within the sheep trial contained a [Ag] of 197 μg/g (approximately 200 μg of gel was used per site during the trial).

Surgical procedures: Details of the techniques for general anaesthesia, tooth extraction, implant placement and histological preparation for sheep have been previously reported (Duncan et al. 2008, Annals of the Royal Australasian College of Dental Surgeons 19:152-156; Duncan et al. 2015, Biomed. Res. Int. 2015:857969; Duncan et al. 2016 Clin. Oral Implants Res. 27(8):975-80; Sharma et al. 2016, J. Mater. Sci, Mater. Med.;27(5):86; Liu et al. 2016, Clin. Oral Implants Res. 27(7):762-70). 1st, 2nd and 3rd mandibular teeth on the left hand lower jaw were extracted under general anaesthetic and the sites allowed to heal for 3 months. One Southern Implants MSC Tapered 14×Ø 3.75 mm implant was placed into the more posterior site. These implants have a blasted surface with a machined (smooth) upper portion which is designed to be easier to clean in the presence of peri-implant infection. One Nobel system implant (diameters 3.75 to 5 mm, length 8.5 to 15 mm, parallel sided configuration) was placed into the anterior site on the left side. Both implants were placed using a two stage protocol, with the implant buried to allow full healing for 2½ months. After 2½ months, the coronal portion of both implants was exposed. Appropriate trephine burs were used to create a 5 mm deep trough around the shoulder of each implant. Cover-screws were removed, a trans-gingival healing abutment was placed and a 3-0 silk ligature tied around the implant threads, positioned within the created surgical intra-bony defect. The surgical site was closed with resorbable sutures. Simultaneously, full-thickness flaps were raised around the 1st and 2nd mandibular premolars on the right side of the lower jaw. A trough was created around the teeth on buccal and lingual surfaces and extending interproximally, using a round bur and a round piezoelectric tip. A 3-0 silk ligature tied around the implant threads, positioned into the created surgical intra-bony defect. The surgical site was closed with resorbable sutures. All sites were irrigated with P. gingivalis pure culture at baseline. All sites were radiographed and were sampled microbiologically using curettes prior to initiation of disease. An additional 4 ewes received immediate implants placed into fresh tooth extraction sites. These animals did not have disease created around either the teeth or implants and thus formed an untreated control group. After 7 weeks of ligature-induced disease, all 20 test and control animals had the ligature removed under general anaesthetic. The teeth and implants had clinical measurements recorded using periodontal or peri-im plant probing using a standard periodontal probe with an 0.5 mm ball end and Williams markings (1, 2, 3, 5, 7, 8, 9, 10 mm). Six sites were measured around each tooth (mesiobuccal and mesiolingual, midbuccal and midlingual, distobuccal and distolingual). Four sites were recorded around implants (mesial, buccal, lingual, distal). Gingival or mucosal recession and pocket depths were recorded separately and combined arithmetrically to determine attachment levels. All sites were radiographed. Microbial flora was sampled using a periodontal curette from around the implants, the premolar teeth and the anterior incisors in all sheep. The 20 sheep were divided into a test group and a control group with 10 sheep in each group. All diseased premolar and implant sites then were scaled and rootplaned using an ultrasonic scaler and hand instruments. The Ag gel formulation was applied using a syringe and blunt cannula to the test animals only. A measured dose of 1 ml was divided evenly around the premolars and the implants. Two test sheep and two control sheep were then euthanased after 1, 2, 4, 8 and 16 weeks. Peri-implant clinical measurements, radiography and microbial sampling were repeated for the four animals at each time point prior to euthanasia. The sheep were then cannulated through the carotid arteries and exsanguinated from the jugular vein with simultaneous perfusion with formalin. The implants and mandibular premolars were removed en bloc with the mandibular bone and further fixed in formalin.

Histology: The implants were separated into individual samples. The premolar teeth were trimmed to a block containing the two mandibular premolars. Micro-CT scans were obtained for the 1 week and 16 weeks specimens using the Skyscan 1172 microCT scanner. All tissues were then dehydrated, embedded in methacrylate resin, sectioned, glued to plastic slides, ground and polished to a final thickness of 90 to 130 μm and stained with MacNeils Tetrachrome & Toluidine blue. In some cases sections were obtained in both bucco-lingual and mesio-distal orientation, but for most the orientation was bucco-lingual. Some slides were counterstained with acid red. The degree of inflammation was described for the most central section from each specimen.

1 week premolars—test specimens: Notching corresponding to the preparation of the intrabony defect was observed on one root surface. The wide periodontal ligament found in sheep (compared with humans) was also observed. Remnants of material that may be silver gel material was observed. The gingiva is closely adapted to the teeth in the region of the cementoenamel junction (note that the enamel extends subgingivally in this species). There was little evidence of inflammation. A cross-section through the furcation region showed residual silver gel lying within the furcation entrance along with some bone debris. The periodontal ligament remained intact through the furcation. Healing of the gingival connective tissue into the notched root and a residual gingival pocket that had been occupied by silver gel could also be seen.

1 week premolars—control specimens: In one specimen the level of crestal bone was markedly more apical on the buccal than the lingual, and this is associated with deposition of new bone on the outer surface of the alveolus and the presence of an open periodontal pocket with degenerating blood clot and an inflammatory infiltrate. A section though the interproximal region between two premolars showed impaction of food debris, residual blood clot and a marked underlying inflammatory infiltrate. In another specimen a longitudinal (sagittal) section through the two premolars, from which a buccolingual specimen had already been removed, demonstrated the interproximal region between first and second premolar. Notches on the mesial surface of the first premolar and in the furcation of the second premolar are both associated with an inflammatory infiltrate. The buccolingual section demonstrated good interproximal healing although residual blood clot was seen within the loosely-organised trabecular bone.

1 week implants—test specimens: The anterior test implants at 1 week were a Nobel Brånemark Mark III 3.75 mm Ø×11.5 mm implant with Tiunite surface and a Nobel Brånemark Mark III 4.0 mm Ø×15 mm implant with Tiunite surface. The posterior implants were Southern Implants MSc 4.0 Ø×13 mm long.

Anterior test implants: The first anterior one-week test implant showed bone loss and inflammatory infiltrate extending for half the length of the implant. The apical part was still osseointegrated. Remnants of bone remained attached to the implant surface after the creation of the intrabony surgical wound using the trephine burs. The other anterior test implant showed bone loss and inflammatory infiltrate extending to near the apex. The implant was still osseointegrated at the apex. Remnants of bone were still present. The apical extent of the epithelial pocket lining could be seen. Similar features could be seen in the mesiodistal section from the same implant as well as remnants of the silver gel. A specimen from the same implant showed extensive inflammation extending to the apex, bony sequestrate and reactionary deposition of new bone on the external surface of the mandible.

Posterior test implants: The first posterior one-week test implant was well integrated for most of the implant length with epithelial downgrowth extending 5 threads (5 mm) apically whilst the trephined defect extended 9 mm apically and was associated with an inflammatory infiltrate of the marginal bone. Some remnant gel was visible within the intrabony lesion. A mesiodistal section showed the distal surface of this implant and the mesial surface of the untreated molar tooth. There appeared to be remnants of the silver gel lying within the pocket but also located within the loose trabecular bone in the adjacent alveolus. The second posterior one-week test implant was also well integrated for most of the implant length. Epithelial downgrowth was limited to the first one to three threads, corresponding to the trephined defect. Inflammatory infiltrate was confined to the more superficial portion of the lesion. A mesio-distal section confirmed that the inflammatory infiltrate extended from the apical extent of the epithelial downgrowth below the base of the trephined defect into the marginal bone. This section also clearly showed the large marrow space with loose cortical bone in the superior portion of the mandible and dense cortical bone in the inferior portion. The white space in the middle of the implant represents the area removed for the buccolingual specimen.

Control specimens: The anterior test implants at 1 week were a Nobel Replace 4.0 mm Ø×13 mm implant with TiUnite surface and a Nobel Brånemark Mark III 3.75 mm Ø×15 mm implant with TiUnite surface. The posterior implants were Southern Implants MSc 4.0 Ø×13 mm long.

Anterior test implants: The first anterior one-week control implant failed before baseline defect creation. The second implant was still present and osseointegrated for 50% of the length of the implant. Considerable unresorbed blood clot occupied the trephined lesion along with some food debris and some bone remnants. External reactionary bone was present. The mesiodistal defect demonstrated the large size of the trephined defect, with the superior cortical bone stopping well short of the implant surface. Clot and debris filled the defect. The marrow space showed the extension of the inflammatory infiltrate into the marrow space.

Posterior test implants: The first posterior one-week control implant was still osseointegrated near the apex, but there was a large pocket full of blood clot, debris and suppuration with inflammation extending to the apex of the implant. This implant was near to failing after one week. The second posterior implant was well osseointegrated, the clear outline of the trephine bure could be seen, as well as some blood clot within the pocket with associated inflammatory infiltrate and limited epithelial downgrowth. A mesiodistal section showed similar features.

16 week Premolars—test specimens: The first test animal showed little evidence of periodontitis, with no marked epithelial downgrowth and only a minor inflammatory infiltrate. A second section from the same animal showed a notch on the buccal surface from the surgical creation of the defect, into which epithelium had healed. The extent of the surgical defect into bone could be seen, with new bone having filled the defect. The second animal showed more recession on the buccal surface, but there was good healing of the original defect. There was little evidence of epithelial downgrowth. The mesiodistal section showed complete healing within the second premolar furcation and a notch on the mesial surface.

Control specimens: The first control animal had persisting periodontal inflammation with an inflammatory cell infiltrate and epithelial downgrowth present on the buccal surface and little evidence of bone regeneration above the defect. A notch in the root surface showed progressive root resorption. A section through the second premolar from this animal also showed a deep buccal pocket and little bone regeneration on the buccal surface. The second control animal showed again a deep pocket adjacent to the notch left when the defect was created, with epithelialisation and little bone regeneration.

16 week implants—test specimens: The anterior test implants at 1 week were a Nobel Brånemark Mark IV 4.0 mm Ø×10 mm long implant with Tiunite surface and a Nobel Brånemark Mark IV 4.0 mm Ø×8 mm long implant with Tiunite surface. The posterior implants were Southern Implants MSc 4.0 Ø×13 mm long.

Anterior test implants: The first anterior 16-week test implant had been lost. The second anterior test implant showed good integration despite being a short implant length. A pocket was present which had some retained material although it was unclear whether this was debris or residual gel. The base of the intrabony pocket was well walled off with connective tissue.

Posterior test implants: The first posterior 16-week test implant remained well integrated for half its length. The intrabony pocket was well walled off by an organised band of connective tissue lined with epithelial. There was some evidence of attempted new bone growth into the defect and little evidence of persistent inflammation, despite residual debris and bone remnants filling the pockets. The second posterior test implant was also well integrated for half its length with a well walled off intrabony pocket lined with epithelium containing orange debris that might well include residual gel and showing evidence of bone regeneration. The mesio-distal specimen showed similar features.

Control specimens: The anterior control implants at 1 week were a Nobel Brånemark Mark III 5.0 mm Ø×11.5 mm implant with Tiunite surface and a Nobel Brånemark Mark III 5.0 mm Ø×13 mm implant with Tiunite surface. The posterior implants were Southern Implants MSc 4.0 Ø×13 mm long.

Anterior control implants: The first anterior 16-week control implant was still present but had completely lost osseointegration and was about to fail. An epithelium-lined inflammatory infiltrate extended completely around the implant. The second anterior control implant had failed and was no longer present.

Posterior control implants: The first posterior 16-week control implant had failed and was no longer present. The second posterior control implant had also failed and was no longer present.

Microbiology: Plaque samples from a premolar and implant region were taken via mechanical scraping methods and placed into 1 mL of sterile PBS buffer. This was performed in vivo at the following stages: i) prior to ligature placement (implant site n=9; premolar site n=11), ii) after inducement of disease (implant n=19; premolar n=19), and iii) at Ag NP-containing hydrogel treatment weeks 1, 2, 4, 8 and 16 (test animals n=2; control animals n=2). Plaque samples were stored at −20° C. until analysis. The defrosted tubes were centrifuged for 1 min at 10,000-12,000 rpm and the supernatant was removed and discarded. A 20 mL volume of InstaGene™ matrix (contains Chelex resin beads) was mixed continually with a magnetic stirrer to maintain resin beads suspension. While stirring, 100 μL of the suspension was added to each plaque pellet using a wide bore pipette. The plaque pellet was incubated at 56° C. for 15-30 min, then vortexed at high speed for 10 s, and subsequently incubated at 100° C. for 8 min. The samples were then vortexed at high speed for 10 s and centrifuged at 10,000-12000 rpm for 2-3 min. The supernatant from each sample, now containing extracted DNA, was collected and placed in 126 separate, sterile Eppendorf tubes. These samples were sent on dry ice to New Zealand Genomics Laboratory (NZGL) at Massey University, New Zealand. Bioanalysis PCR and high throughput Illumina sequencing was performed using a 16S universal primer by NZGL as described as follows.

Amplicon PCR: DNA samples were amplified through the use of PCR thereby generating a large quantity of the 16S region for further analysis. For each sample, the following reaction (Table 3) was set up in a separate well (0.2 μL) of a 96 well plate. The full length primer sequences, using IUPAC nucleotide nomenclature, were as follows:

16S Amplicon PCR Forward Primer = 5′
(SEQ ID NO. 1)
TCGTCGGCAGCGTCAGATGTGTATAAGAGACAGCCTACGGGNGGCWGCAG
16S Amplicon PCR backward Primer = 5′
(SEQ ID NO. 2)
GTCTCGTGGGCTCGGAGATGTGTATAAGAGACAGGACTACHVGGGTATCT
AATCC

TABLE 3
Reaction quantities for Amplicon PCR mixture

| | Volume (μL) |
|---|---|
| Microbial Genomic DNA (5 ng/μL in 10 mM Tris pH 8.5) | 2.5 |
| Amplicon PCR forward primer 1 μM | 5 |
| Amplicon PCR backward primer 1 μM | 5 |
| 2 x KAPA HiFi HotStart Ready Mix | 12.5 |
| Total | 25 |

The plate was then sealed using Microseal ‘A’ film, placed in a thermal cycler and PCR was performed using the following program: 95° C. for 3 min; 25 cycles of: 95° C. for 30 s, 55° C. for 30 s, 72° C. for 30 s; 72° C. for 5 minutes and then samples were held at 4° C. until the next step. Subsequently, 1 μl of the PCR product was then run on a Bioanalyzer DNA 1000 chip to verify size.

PCR clean-up 1: Free primers and primer dimer species were removed using AMPure XP beads. The AMPure XP beads were maintained at room temperature prior to immediate placement in the PCR mixture. The 96-well microplate from “Amplicon PCR” was centrifuged at 1,000×g at 20° C. for 1 min to collect condensation from the top of the plate. Subsequently, the Microseal ‘A’ film was carefully removed. The AMP XP beads were vortexed for 30 s. Using a multichannel pipette, 20 μL of the AMP XP beads were added to each well of the PCR 96-well plate. Using separate tips for each column, the entirety of the well volumes were gently pipetted up and down 10 times. The PCR well plate was maintained at room temperature for 5 min. The plate was then placed on a magnetic stand for 2 min until the supernatant become clarified as a result of bead sedimentation. The supernatant was removed and discarded. Care was taken not to remove any of the pelleted material. With the PCR plate still on the magnetic stand, 200 μL of freshly prepared 80% ethanol was added to each of the wells and was then left for 30 s. The supernatant was then carefully removed and discarded. The ethanol wash was performed twice. However, on the second wash a final step was introduced where a fine pipette was used to remove the excess ethanol. The contents of the plate were then allowed to air dry for 10 min. The PCR plate was removed from the magnetic stand and using a multichannel pipette, 52.5 μL of 10 mM Tris pH 8.5 was added to each well. The contents of the wells were then pipetted up and down 10 times, using fresh tips for each column, ensuring the beads were re-dispersed, and the plate was then maintained at room temperature for 2 min. The PCR plate was placed again on the magnetic stand for 2 min until the supernatant became clarified as a result of bead sedimentation. Afterwards, from each sample, 50 μL of the supernatant was carefully transferred to a new well within a fresh 96-well PCR plate. The new PCR plate was then stored at −15° C. to −25° C. for up to a week prior to the index PCR step.

Index PCR: Index PCR only required use of 5 μL of the Amplicon PCR product from the previous step. Therefore, 45 μL was stored in the PCR well plate at −15° C. to −25° C.

A 5 μL volume was transferred from the ‘PCR clean up’ plate to a new PCR plate. The following volumes (Table 4) of materials were added to the wells to make up a total volume of 25 μL.

TABLE 4
Reaction quantities for index PCR mix

| | Volume (μL) |
|---|---|
| DNA | 5 |
| Nextera XT Index Primer 1 (N7xx) | 5 |
| Nextera XT Index Primer 2 (S5xx) | 5 |
| 2x KAPA HiFi Hotstart ReadyMix | 25 |
| PCR Grade H₂O | 10 |
| Total | 50 |

The mixture was gently pipetted up and down 10 times. The plate was then covered with a Microseal ‘A’ film and was then centrifuged at 1,000×g at 20° C. for 1 min. PCR was performed on a thermal cycler using the following program: 95° C. for 3 min; 8 cycles of: 95° C. for 30 s, 55° C. for 30 s, 72° C. for 30 s; 72° C. for 5 min; the plate was then held at 4° C. until the next step.

PCR clean-up 2: The second PCR clean-up was performed on the Index PCR product as described in the ‘PCR clean up 1’, with the following modifications. A volume of 56 μL AMPure XP beads was transferred into each well of the Index PCR plate. After two steps of ethanol washing, a volume of 27.5 μL of 10 mM Tris, pH 8.5, was added to the beads in the Index PCR plate. Afterwards, from each sample, 25 μL of the supernatant was carefully transferred to a new well within a fresh 96-well PCR plate. The PCR plate was then stored for up to 1 week at −12° C. to −25° C. before the library quantification, normalization and pooling processes were performed.

Library Denaturing and MiSeq Sample Loading: The final pooled DNA (4 nM, 5 μg) was placed in a microcentrifuge tube with 0.2N NaOH (5 μl). The microcentrifuge tube was vortexed and centrifuged at 280×g at 20° C. for 1 min. The tube was then maintained at room temperature for 5 min to allow for the DNA to denature into single strands. Pre-chilled hybridization buffer (HT1; 990 μL) was then added to the 10 μL volume of denatured DNA in the microcentrifuge tube. Thus, the microcentrifuge tube contained a 20 pM denatured library in 1 mM NaOH, and was placed on ice until the final dilution was performed.

Dilute Denatured DNA: The denatured DNA was then diluted to the desired concentration using Table 5.

TABLE 5
Dilution quantities for respective Illumina analysis concentrations

| Final Conc. | 2 pM | 4 pM | 6 pM | 8 pM | 10 pM |
|---|---|---|---|---|---|
| 20 pM denatured library | 60 μL | 120 μL | 180 μL | 240 μL | 300 μL |
| Pre-chilled HT1 | 540 μL | 480 μL | 420 μL | 360 μL | 300 μL |

The tube of diluted DNA was then inverted several times, pulse centrifuged and stored on ice.

Denature and Dilution of PhiX Control: PhiX library (10 nM; 2 μL) was combined with Tris pH 8.5 (10 mM; 3 μL) to make an overall PhiX library dilution of 4 nM. The diluted PhiX (5 μL) was further combined with 0.2 N NaOH (5 μL) into a microcentrifuge tube. The microcentrifuge tube was vortexed and then maintained at room temperature for 5 min to allow for denaturing of the PhiX into single strands. The denatured PhiX library (10 μL) was then added to pre-chilled HT1 (990 μL). The concentration of the denatured PhiX was now at 20 pM and was further diluted according to Table 5. The tube of diluted DNA was then inverted several times, pulse centrifuged and stored on ice.

Combine Amplicon Library and PhiX Control: The denatured and diluted PhiX control (30 μL) was combined with the denatured and diluted amplicon library (570 μL). The microcentrifuge tube containing the combined libraries was stored on ice prior to heating which was only performed before immediately loading the sample into the MiSeq v3 reagent cartridge. The MiSeq reagent cartridge is a single use consumable, containing the necessary clustering and sequencing reagents for one flow cell. The microcentrifuge tube was place in a heat block at 96° C. for 2 min. The tube was then inverted 1-2 times and placed into an ice water bath for 5 min.

MiSeq Reporter Metagenomics Workflow: After the samples were loaded and sequencing was performed on the MiSeq, the classification of organisms from the V3-V4 amplicon was performed using a 16S rRNA data (Greengenes database; http://greengenes.lbl.gov/). The classification determines the following taxonomic levels: kingdom, phylum class, order, family, genus and species. In this case, due to limitations of the method used, the lowest taxa available for reliable determination was genus.

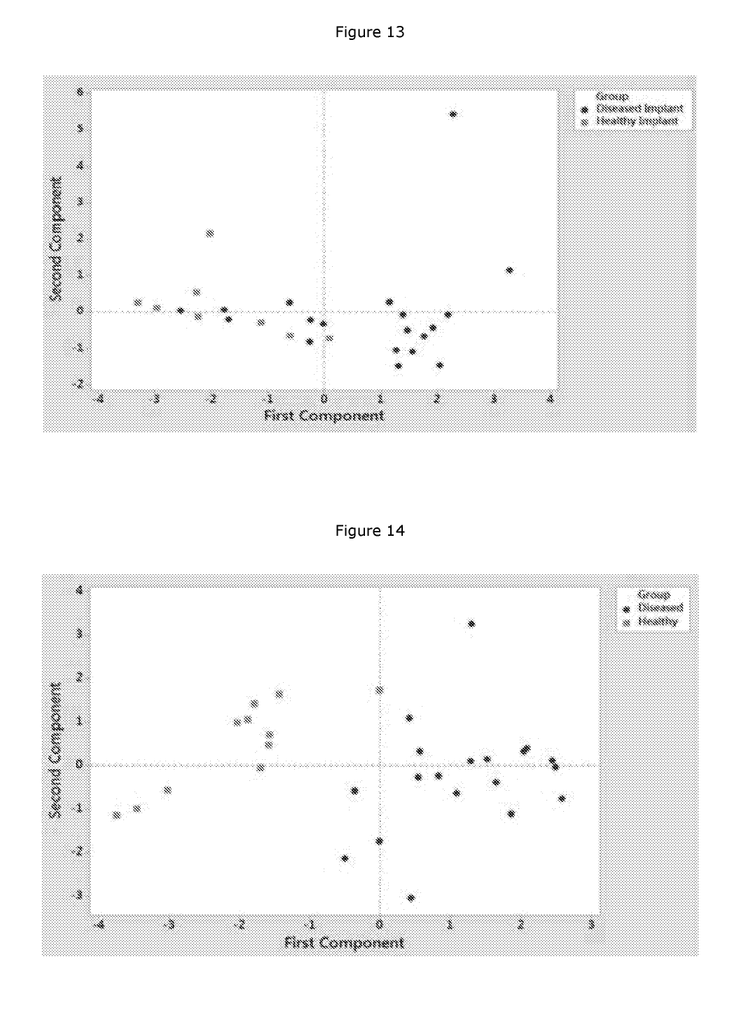

Microbiology Results: The microbiology data sets obtained for baseline and diseased samples from both the implant sites and the premolar sites were separately subjected to principal components analysis (PCA) using Minitab 17 Statistical software, and the scores plots are shown in FIGS. 13 and 14, respectively. The scores plots show clustering of the samples in component space, with both implant site samples and premolar site samples forming two groups, separating out the baseline and diseased samples. As the number of sheep differed between baseline samples (N=9 and N=11) and diseased samples (N=19 and N=19), the sample clustering can be considered a more reliable suggestion of differentiation of the microorganism profile of baseline and diseased sheep, although outliers could still be observed in the PCA analysis.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

1. A gel comprising:
(i) nano-sized particles of metallic silver (Ag);
(ii) a polymer comprising carboxylate groups;
(iii) carboxylate molecules comprising at least one group capable of binding to Ag; and
(iv) metal ions;
wherein:
at least some of the Ag particles are bound to each other through a carboxylate-metal ion bridge as shown in formula (I):
Ag—X-M-X—Ag (I)
where X is a carboxylate molecule, and M is a metal ion; and
at least some of the Ag particles are bound to a polymer chain through a carboxylate-metal ion bridge as shown in formula (II):
Ag—X-M-Y (II)
where X is a carboxylate molecule, M is a metal ion, and Y is a carboxylate group of the polymer.

2. A gel as claimed in claim 1, wherein the polymer is a polysaccharide.

3. A gel as claimed in claim 1, wherein the polymer is selected from the group consisting of: alginic acid, hyaluronic acid, polyglutamic acid, polygalacturonic acid, and carboxymethyl cellulose.

4. A gel as claimed in claim 1, wherein the at least one group capable of binding to Ag is a thiol group or an amine group.

5. A gel as claimed in claim 1, wherein the carboxylate molecules are alkylcarboxylate molecules.

6. A gel as claimed in claim 5, wherein the alkylcarboxylate molecules are straight chain or branched, cyclic or acyclic, aromatic or non-aromatic C₄-C₁₀alkylcarboxylate molecules.

7. A gel as claimed in claim 1, wherein the carboxylate molecules are selected from the group consisting of: 6-mercaptohexanoic acid, 8-mercaptooctanoic acid, mercaptosuccinic acid, 4-mercaptobenzoic acid, 4-mercaptophenylacetic acid, lipoic acid, dihydrolipoic acid, glutathione, penicillamine, 5-(4-amino-6-hydroxy-2-mercapto-5-pyrimidinyl)pentanoic acid, and 2-mercapto-4-methyl-5-thiazoleacetic acid.

8. A gel as claimed in claim 1, wherein the metal ions are divalent metal ions.

9. A gel as claimed in claim 8, wherein the divalent metal ions are calcium, zinc, magnesium or strontium ions.

10. A gel as claimed in claim 1, having a Ag concentration in the range 230 to 1025 μg/mL.

11. A method of preparing a gel as claimed in claim 1 comprising the steps:
(i) treating a Ag salt with a reducing agent to form nano-sized particles of Ag;
(ii) treating the particles of Ag with a carboxylic acid to form a solution of Ag-carboxylate molecules; and
(iii) treating the Ag-carboxylate molecules with a polymer in the presence of one of more metal ions to form the gel.

12. A method of treating of preventing a microbial infection comprising applying a gel as claimed in claim 1.

13. The method as claimed in claim 12, wherein the microbial infection is a bacterial infection.

14. The method as claimed in claim 13, wherein the bacterial infection is caused by Streptococcus mutans, Streptococcus mitis, Streptococcus gordonii, Enterococcus faecalis, Staphylococcus oxford, Pseudomonas aeruginosa or Escherichia coli.

15. The method as claimed in claim 12, wherein the infection is periodontitis or peri-implantitis.

16. The method as claimed in claim 12, wherein the microbial infection is a fungal infection.

17. The method as claimed in claim 16, wherein the fungal infection is a Candida infection.

18. The method as claimed in claim 16, wherein the infection is denture stomatitis.

19. The method as claimed in claim 13, wherein the infection is periodontitis or peri-implantitis.

20. The method as claimed in claim 14, wherein the infection is periodontitis or peri-implantitis.

21. The method as claimed in claim 17, wherein the infection is denture stomatitis.
